(19) United States
(12) Patent Application Publication
(10) Pub. No.: US 2020/0276200 A1
(43) Pub. Date: Sep. 3, 2020

(54) TREATMENT OF CNS AND DEVELOPMENTAL DISORDERS USING HIGH-DOSE 5-FORMYL-(6S)-TETRAHYDROFOLATE

(71) Applicant: Clark Gerald Sullivan, New York, NY (US)

(72) Inventor: Clark Gerald Sullivan, New York, NY (US)

(21) Appl. No.: 16/664,797

(22) Filed: Oct. 26, 2019

Related U.S. Application Data
(60) Provisional application No. 62/911,282, filed on Oct. 6, 2019.
(60) Provisional application No. 62/811,562, filed on Feb. 28, 2019.

(51) Int. Cl. A61K 31/519; A61K 9/00; A61K 9/08

(52) U.S. Cl. CPC A61K 31/519; A61K 9/0056; A61K 47/12; A61K 9/006; A61K 9/08

(57) ABSTRACT

Methods for treating developmental or chronic cerebral nervous system disorders using 5-formyl-(6S)-tetrahydrofolate and pharmaceutically acceptable dosage forms therefor are provided.

### FIELD OF THE INVENTION

The present invention relates to the treatment of developmental disorders and chronic disorders of the cerebral nervous system (CNS) associated with perturbed folate metabolism, using high-dose 5-formyl-(6S)-tetrahydrofolate and pharmaceutically acceptable salts thereof.

### BACKGROUND OF THE INVENTION

As reported in a recent review by Zheng 2018, folates is a generic term referring to a large family of compounds consisting of a 2-amino-4-hydroxy-pteridine ring, linked by a methylene (CH2) group to a p-aminobenzoyl moiety, which is in turn linked through an amide bond to the α-amino group of a monoglutamate or poly-γ-glutamate. One-carbon (1C) units can be attached to N5, N10, or both. On the other hand, the name folic acid is reserved for the synthetic form with the fully oxidized pteridine ring and no 1C substitution. The central entity in the folate family of compounds is known as tetrahydrofolate or THF, and its chemical structure is reproduced below:

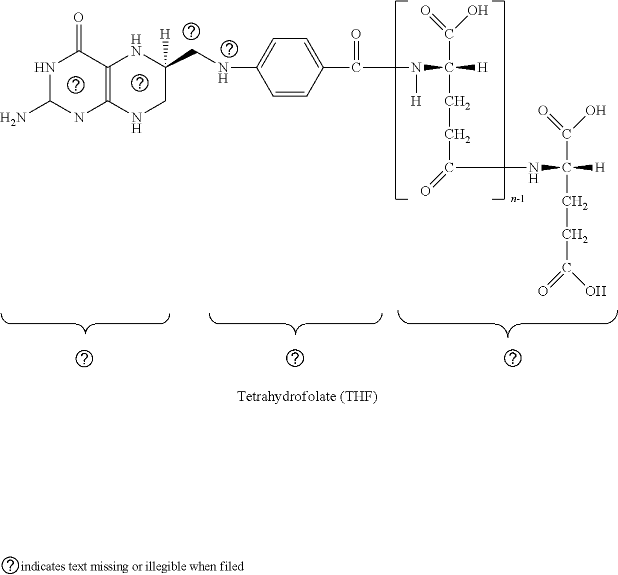

The number of distinct molecular species within the folate family could be as many as 150, due to the combinatorial effect of varying 1C and pteridine oxidation states and varying polyglutamate chain lengths. Nevertheless, typically <50 species are detectable in natural animal and plant sources, with the pteridine ring predominantly in the most reduced, tetrahydro state, N5 and N10 either unsubstituted or substituted with one of six different 1C units, and the polyglutamate chain length being predominantly five to eight. Commercially available folates include pteroylglutamic acid (PGA or folic acid), 5-formyl-tetrahydrofolate (folinic acid or leucovorin), 5-formyl-(6S)-tetrahydrofolate (L-folinic acid or 5-methyl-(6S)-THF), 5-methyl THF (5-methyl-tetrahydrofolate or methylfolate), and 5-methyl-(6S)-THF (5-methyl-(6S)-tetrahydrofolate or (6S)-methylfolate), in various salt and acid forms.

As shown in FIG. 1, the central folate for all folate metabolism, to which all folates are eventually converted, is tetrahydrofolate. As reported in a recent review by Stover 2017, tetrahydrofolates (THF) serve as cofactors that carry one-carbon units at three different oxidation states, and function in concert with other B-vitamins including vitamin B12, vitamin B6, riboflavin, and niacin in a metabolic network known as folate-mediated one-carbon metabolism. One-carbon metabolism occurs in the mitochondria, the cytosol, and the nucleus. One-carbon metabolism in the cytosol is necessary for the de novo synthesis of purines and for the remethylation of homocysteine to methionine. Methionine can be converted to S-adenosylmethionine, which is a one-carbon donor for numerous methylation reactions, including DNA methylation and neurotransmitter synthesis and degradation. The de novo thymidylate biosynthesis pathway functions in the nucleus and is essential for DNA synthesis and stability. These THF-dependent critical biochemical functions are the basis of fundamental cellular processes that when disrupted by imbalanced nutrient supply manifest in disease. Nutrition and related genetic epidemiological studies in addition to randomized controlled trials implicate impaired folate metabolism in several pathologies including neural tube defects, neurodegenerative and neuropsychiatric diseases and cancer.

Biomarkers of folate status and function include decreased plasma and/or red blood cell levels of the individual vitamins, as well as depressed S-adenosyl-methionine levels and elevated plasma total homocysteine and S-adenosylhomocysteine. Consequently, a resulting decrease in the ratio of S-adenosylmethionine to S-adenosylhomocysteine is associated with hypomethylated DNA which affects gene expression and DNA stability and alters synthesis of small molecules including neurotransmitters. Impaired one carbon metabolism also depresses thymidylate synthesis, resulting in uracil mis-incorporation and accumulation into DNA leading to lower rates of cell division and cell death, which can compromise neurogenesis.

Many nutrients and metabolites are transported and concentrated up to several-fold higher in cerebral spinal fluid (CSF) than plasma concentrations by specific transport systems through the blood brain barrier (BBB) or choroid plexus. The ratio of CSF/serum for such nutrients can be used as a measure of brain nutrient status, and report on physiological processes such as nutrient transport and degradation and also report on the adequate functioning of the BBB. Folate is concentrated 1.5-3-fold across the BBB, and thus transport across this concentration gradient requires adenosine triphosphate (ATP). Spector 1989 reports that the choroid plexus system normally maintains CSF plasma concentrations of methyltetrahydrofolate at approximately 80 nM, or roughly four times higher than normal plasma concentrations of approximately 20 nM.

The critical role of the BBB in maintaining adequate brain folate status has been demonstrated in pediatric patients with in-born errors of metabolism. Low CSF folate can result from genetic loss-of-function mutations that affect folate utilization, energy metabolism, or folate transport across the BBB, and the resultant cerebral folate deficiency is associated with neurological and neuropsychiatric symptoms. Inborn errors contributing to low CSF folate include folate receptor mutations, folate transport mutations, and mitochondrial DNA depletions.

As reported by Ramaekers 2002, two folate transport mechanisms have been identified in man, the reduced folate carrier 1 (RFC1) and the folate receptor proteins (FR). Both mechanisms involve the transport of folates from plasma to the cell interior, as well as folate transport across the placenta, intestinal, and BBB. RFC1 represents and integral membrane protein operating only at relatively high folate concentrations within the micromolar range that is driven by anionic gradients. A genetic defect of the RFC1 system is thought to be the most likely cause of a rare hereditary disease of intestinal folic acid absorption, causing a failure to thrive, macrocytic anemia, microcephaly, developmental delay, ataxia, extrapyramidal movement disorders and convulsions with intracranial calcifications. The membrane-attached folate receptors (FR1 and FR2) possess high affinity for folate in the nanomolar range and therefore are able to bind physiological levels of folate. FR1 (a/k/a folate binding protein 1 or folate receptor alpha) shows a higher affinity for 5-methyl THF compared with FR2 (a/k/a folate binding protein 2 or folate receptor beta)

Stover 2017 further reports that rare genetic mutations in genes encoding proteins involved in folate transport, including the folate receptor alpha (FRa) and the proton-coupled folate transporter (PCFT), result in cerebral folate deficiency, as do mitochondrial DNA depletion syndromes associated with defective oxidative phosphorylation, and the Kearnes-Sayre syndrome. In addition to mutations in genes that are involved in brain folate transport and accretion, mutations in folate metabolism genes including methylenetetrahydrofolate reductase (MTHFR), dihydrofolate reductase and mutations in genes that result in brain serine depletion also affect brain folate levels. Serine is a major source of one-carbon groups for folate-mediated one carbon metabolism. Deficiencies in these enzymes likely increase rates of folate catabolism and turnover because unstable forms of folate accumulate when enzyme activity is impaired. Alternatively, brain folate deficiency can result from lack of ATP (energy) required to maintain the energy gradient across the BBB.

Adults can also be at risk for brain nutrient deficiencies, including folate deficiency because of diminished BBB function. Adult-onset BBB dysfunction can be caused by, mitochondria depletion, as well as chronic conditions and diseases including inflammation, hypoxia, diabetes mellitus, hypertension, cerebrovascular ischemia, acute kidney injury, viral infection, parasitic infection, and Alzheimer's Disease. Chronic disease can accelerate age-associated deterioration of BBB functions including nutrient transport, which can lead to nutritional deficiencies that are isolated to the central nervous system.

Autoimmunity is an emerging risk factor for late-onset cerebral folate deficiency. Autoantibodies against the folate receptor alpha (FRa) that block folate transport across the blood-brain barrier have been correlated with low CSF folate. The presence of autoantibodies against FRa and cerebral folate deficiency are seen in some autism spectrum disorders, depression, schizophrenia and seizures. Common genetic variants can also contribute to low CSF folate in disease. The best-characterized gene variant is the 677C to T MTHFR polymorphism. The MTHFR variant has dual effects on FOCM that include both its effect on folate status and its impacts on MTHFR catalysis, including its role in providing (6S) 5-methyltetrahydrofolate for the homocysteine remethylation pathway and cellular methylation.

The recommended dietary allowance (RDA) for folate dietary equivalents is 0.4 mg/day for healthy adults, but this recommended level of intake may not be sufficient for some individuals with chronic CNS disorders. Cerebral folate deficiency, caused by folate receptor autoantibodies or germline mutations in FOLR1 or SLC46A1, is manifested in neurological impairments and shows some resemblance to severe MTHFR deficiency. Blood folate levels are poor indicators of cerebrospinal fluid (CSF) folate levels, but elevated intake of folate can correct low CSF folate levels in patients with in-born errors of metabolism or other CSF transport disorders. Parenteral folinic acid (5 mg, twice weekly) or oral folinic acid (0.5-20 mg/kg/day) is used, tolerated and normalizes CSF folate levels with improvements in neurological outcomes and white matter morphology. Exposure to high levels of reduced forms of folate (such as folinic acid) can overcome transport defects that may arise and restore CSF folate levels, physiological function and improve clinical outcomes.

In spite of this important role of folates during neurological development and in chronic CNS disorders, very little is known about the differences in biochemical metabolism and clinical outcomes among various types of folates. What is needed are studies to elucidate differences in various folate forms and the metabolism of those forms, and to determine which are most efficiently taken up by tissues with high demand for folates and which are best suited to treat specific medical conditions.

### SUMMARY OF INVENTION

Several unexpected advantages have been discovered from the administration of high dose 5-formyl-(6S)-tetrahydrofolate, when compared to other common folates such as folic acid, folinic acid, and 5-methyl-(6S)-tetrahydrofolate (“5-MTHF”), particularly in the treatment of chronic CNS disorders and other developmental disorders caused by perturbed folate metabolism in the brain or developing fetus. Compared to other common forms of folate, including folic acid, folinic acid and 5-MTHF, high doses of 5-formyl-(6S)-THF produce higher concentrations of total folates in the CNS, and better distributions of folate species including tetrahydrofolates which are essential to DNA synthesis, cellular replication, and neurogenesis. Tetrahydrofolates are also important based on their reported superiority as antioxidants compared to other folate species, as reported by Rezk 2003. These results are particularly pronounced in pregnant mammals and the developing fetus, where 5-formyl-(6S)-THF results in substantially higher folate uptake in critical organs such as the ovary, placenta and brain.

Furthermore, in contrast to prior art teachings that minimal amounts of the un-natural D-isomer of folinic acid are absorbed into cells when folinic acid is administered, the inventors have unexpectedly discovered significant uptake of the D-isomer in critical tissues such as the ovary and brain, particularly when transport through the FR-alpha receptor is compromised. This preferential uptake of the D-isomer from folinic acid is not without consequence, given the toxicity reported by FDA for the D-isomer in its pharmacology review for Fusilev® (NDA 20-140), and reports by Bertrand 1989 of how that the D-isomer interferes with absorption of natural folates in the L-configuration.

These discoveries have particular applicability to children with autism, particularly young children with expressive communication deficits, even those children with normal CSF folate concentrations. Thus, in a first principal embodiment the invention provides a method of improving MSEL expressive communication scores in a child less than 6 years of age, wherein the child is characterized by: (a) a moderate level of autism under ADOS-2, and (b) a normal CSF folate concentration of from 39.7 nmol/l to 174 nmol/l; comprising orally administering to the child from a preservative free dropper from 5 to 25 35-60 mcl drops per day of a pharmaceutical formulation comprising a preservative-free liquid solution of 5-formyl-6(s)-tetrahydrofolate or a pharmaceutically acceptable salt thereof, at a concentration of from 20 to 30 mcg/ml, that has an absolute bioavailability of 5-formyl-6(s)-tetrahydrofolate greater than or equal to 92%.

More generally, in a second principal embodiment, the invention provides a method of improving MSEL expressive communication scores in a child less than 6 years of age having a developmental or chronic cerebral nervous system disorder comprising orally administering to the child a pharmaceutical formulation comprising a therapeutically effective amount of 5-formyl-6(s)-tetrahydrofolate or a pharmaceutically acceptable salt thereof.

The invention also can be used to improve other measures of intellectual functioning. Thus, in a third principal embodiment the invention provides a method of improving a measure of social or intellectual functioning in a child less than 6 years of age having a developmental or chronic cerebral nervous system disorder comprising orally administering to the child a pharmaceutical formulation comprising a therapeutically effective amount of 5-formyl-6(s)-tetrahydrofolate or a pharmaceutically acceptable salt thereof, wherein the measure of intellectual functioning is selected from: (a) an MSEL cognitive index score, or an MSEL score in the expressive language, receptive language, fine motor, visual reception, or gross motor domain; (b) a BOSCC social communication score; (c) a global score under the ABC or an ABC score in the irritability, social withdrawal, stereotypic behavior, hyperactivity, or inappropriate speech domain; (d) a global severity score under the ADOS-2 or a reciprocal social interaction or communication sub-score under the ASOS-2; or (e) a VABS total score or a VABS score in the communication, daily living, social skills, motor skills, or adaptive behavior domain.

The invention is further premised on the pharmacokinetics surprising observed for the formulations of the present invention, when ingested as an oral aqueous solution, and their utility treating developmental and chronic cerebral nervous system disorders. Thus, in a fourth principal embodiment the invention provides a method of treating a developmental or chronic cerebral nervous system disorder in a child comprising orally administering to the child a pharmaceutical formulation comprising an aqueous solution when ingested of a therapeutically effective amount of 5-formyl-6(s)-tetrahydrofolate or a pharmaceutically acceptable salt thereof.

Still further embodiments are premised on the prevention of development and chronic cerebral nervous disorders in a developing fetus, by administering the disclosed formulations to a pregnant mother. Thus, in a fifth principal embodiment the invention provides a method of treating or preventing a developmental or chronic cerebral nervous system disorder in the unborn child of a maternal human in need thereof, comprising orally administering to the maternal human a pharmaceutical formulation comprising a therapeutically effective amount of 5-formyl-6(s)-tetrahydrofolate or a pharmaceutically acceptable salt thereof.

A still further embodiment relates to a drug/device combination specially adapted for administering the formulations of the present invention to children who are particularly young. Thus, in a sixth principal embodiment the invention provides a drug product comprising a multi-dose dropper comprising from 5 to 50 ml of an aqueous oral solution of 5-formyl-(6S)-tetrahydrofolate or a pharmaceutically acceptable salt thereof, wherein the concentration of 5-formyl-(6S)-tetrahydrofolate in the solution is from 20 to 30 mg/ml based on the weight of the anhydrous free acid, and each drop dispensed from the dropper comprises from 35 to 150 microliters of solution.

Additional advantages of the invention are set forth in part in the description which follows, and in part will be obvious from the description or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

A better understanding of the present invention can be obtained when the following detailed description of the disclosed embodiments is considered in conjunction with the following drawing, in which:

FIG. 1 is a depiction of the folate cycles and their connection to other critical metabolic processes, as reported in the prior art.

FIG. 2 is a sample questionnaire for the Clinician Global Impression of Verbal Communication (CGI-VC) useful for anchoring clinically meaningful improvements under the MSEL expressive or receptive language domains.

FIG. 3 is a sample questionnaire for the Clinician Global Impression of Language Ability (CGI-LA) useful for anchoring clinically meaningful improvements under the MSEL expressive or receptive language domains.

FIG. 4 is a sample questionnaire for the Clinician Global Impression of Change (CGI-C) useful for anchoring clinically meaningful improvements under the MSEL expressive or receptive language domains.

FIG. 5 is a sample questionnaire for the Caregiver Global Impression of Verbal Communication (CaGI-VC) useful for anchoring clinically meaningful improvements under the MSEL expressive or receptive language domains.

FIG. 6 is a sample questionnaire for the Caregiver Global Impression of Language Ability (CaGI-LA) useful for anchoring clinically meaningful improvements under the MSEL expressive or receptive language domains.

FIG. 7 is a sample questionnaire for the Caregiver Global Impression of Change (CaGI-C) useful for anchoring clinically meaningful improvements under the MSEL expressive or receptive language domains.

FIG. 8 is a representative Cumulative Distribution Function “(CDF”) for evaluating clinically meaningful improvements.

FIG. 9 is a representative Kernel Density Plot (“KDP”) for evaluating clinically meaningful improvements.

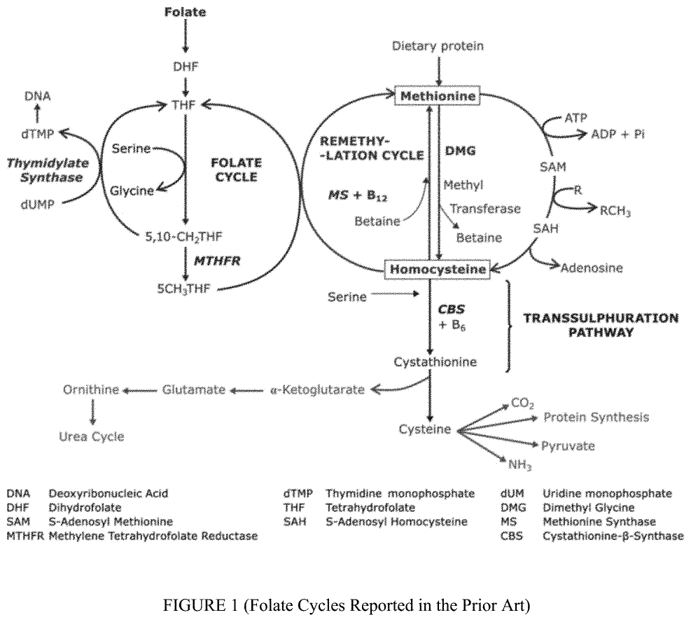

### DETAILED DESCRIPTION

The present invention may be understood more readily by reference to the following detailed description of preferred embodiments of the invention and the Examples included therein.

### Definitions and Use of Terms

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

As used in the specification and claims, the singular forms a, an, and the include plural references unless the context clearly dictates otherwise. For example, the term “a specification” refers to one or more specifications for use in the presently disclosed methods and systems. “An ingredient” includes mixtures of two or more such ingredients, and the like. The word “or” or like terms as used herein means any one member of a particular list and also includes any combination of members of that list.

As used in this specification and in the claims which follow, the word “comprise” and variations of the word, such as “comprising” and “comprises,” means “including but not limited to,” and is not intended to exclude, for example, other additives, components, integers or steps. When an element is described as comprising one or a plurality of components, steps or conditions, it will be understood that the element can also be described as “consisting of” or “consisting essentially of” the component, step or condition, or the plurality of components, steps or conditions.

When used herein the term “about” will compensate for variability allowed for in the pharmaceutical industry and inherent in products in this industry, such as differences in product strength due to manufacturing variation and time-induced product degradation, as well as differences due to waters of hydration and different salts. The term allows for any variation, which in the practice of good manufacturing practices, would allow the product being evaluated to be considered therapeutically equivalent or bioequivalent in humans to the recited strength of a claimed product. In one embodiment the term allows for any variation within 5% of the recited specification or standard. In one embodiment the term allows for any variation within 10% of the recited specification or standard.

When published test methodologies and diagnostic instruments are referred to herein, it will be understood that the test methodology or diagnostic instrument is performed based on the version in effect on Jan. 1, 2018, unless otherwise stated to the contrary herein.

When percentages, concentrations or other units of measure are given herein, it will be understood that the units of measure are weight percent unless otherwise stated to the contrary.

When ranges are expressed herein by specifying alternative upper and lower limits of the range, it will be understood that the endpoints can be combined in any manner that is mathematically feasible. Thus, for example, a range of from 50 or 80 to 100 or 70 can alternatively be expressed as a series of ranges of from 50 to 100, from 50 to 70, and from 80 to 100. When a series of upper bounds and lower bounds are related using the phase “and” or “or”, it will be understood that the upper bounds can be unlimited by the lower bounds or combined with the lower bounds, and vice versa. Thus, for example, a range of greater than 40% and/or less than 80% includes ranges of greater than 40%, less than 80%, and greater than 40% but less than 80%. Unless otherwise specified by the term “between,” the boundaries of the range (lower and upper ends of the range) are included in the claimed range.

When an element of a process or thing is defined by reference to one or more examples, components, properties or characteristics, it will be understood that any one or any combination of those components, properties or characteristics can also be used to define the child matter at issue. This might occur, for example, when specific examples of an element are recited in a claim (as in a Markush grouping), or an element is defined by a plurality of characteristics. Thus, for example, if a claimed system comprises element A defined by elements A1, A2 and A3, in combination with element B defined by elements B1, B2 and B3, the invention will also be understood to cover a system defined by element A without element B, a system in which element A is defined by elements A1 and A2 in combination with element B defined by elements B2 and B3, and all other possible permutations.

In the context of the present invention insofar as it relates to any of the disease conditions recited herein, the term “treatment” means to reduce the occurrence of a symptom or condition, or to relieve or alleviate at least one symptom associated with such condition, or to slow or reverse the progression of such condition, or to manage or affect the metabolic processes underlying such condition. Within the meaning of the present invention, the term also denotes to arrest, or to “prevent,” i.e. to delay the onset (i.e., the period prior to clinical manifestation of a disease) and/or reduce the risk of developing or worsening a disease. When a person with the condition is affirmatively recited, the term will be understood to require relief or alleviation of at least one symptom associated with the condition.

The phrase “acceptable” as used in connection with compositions of the invention, refers to molecular entities and other ingredients of such compositions that are physiologically tolerable and do not typically produce untoward reactions when administered to a child (e.g., a mammal such as a child). “Pharmaceutically acceptable” means that which is useful in preparing a pharmaceutical composition that is generally safe, non-toxic and neither biologically nor otherwise undesirable and includes that which is acceptable for veterinary use as well as child pharmaceutical use.

“Absolute bioavailability” refers to the AUC_{0-inf} achieved by an oral formulation of 5-formyl-(6S)-tetrahydrofolate of the present invention relative to the AUC_{0-inf} achieved by in intravenous administration of the same dose of 5-formyl-(6S)-tetrahydrofolate, looking at all active L-folates appearing in the bloodstream. Unless otherwise specified, the absolute bioavailability is determined by reference to a 12.5 mg dose of 5-formyl-(6S)-tetrahydrofolate.

Where titers of blocking antibodies are expressed herein, the titer obtained by measuring the blocking of radiolabeled folic acid binding to a known amount of purified FRa from child milk as described by Berrocal-Zaragoza M I et al, 2009, or a comparable method, is intended. Blocking autoantibodies prevent the binding of [3H]folic acid to the folate receptor and the autoantibody titer is expressed as pmol receptor blocked/mL of serum. The blocking antibodies could be either IgG (indicative of past infections) or IgM (indicative of current infections) and this method does not identify any specific antibody type.

Where titers of binding antibodies are expressed herein, the titer obtained by the ELISA-based measurement of IgG immunoglobulins that bind to epitopes on purified apo-FRa from child milk described by Molloy A M, et al., 2009, or a comparable method, is intended. The assay only identifies IgG autoantibodies of either the blocking or binding type and does not identify IgM antibodies.

When concentrations or doses of 5-formyl-(6S)-tetrahydrofolate and other folates are expressed herein, it will be understood that the concentration or dose is based on the weight of the anhydrous free acid. When concentrations of folates in bodily fluids are expressed herein it will be understood to refer to concentrations of folic acid and the D- and L-forms of 5-methyl-THF unless expressly stated to the contrary.

5-formyl-(6S)-tetrahydrofolate or a pharmaceutically acceptable salt thereof, when referenced in a pharmaceutical formulation, refers to 5-formyl-(6S)-tetrahydrofolate in the absence of the 6R-enantiomer. I.e., the formulation comprises less than 1% or 0.5% of the 6R-enantiomer based on the weight of the 5-formyl-(6S)-tetrahydrofolate.

Normal CSF (cerebral spinal fluid) concentrations of folate are determined by reference to the study published in Hyland 1992, using the test methodology described therein. In particular, normal CSF concentrations refers to CSF folate concentrations ranging from 39.7 nmol/l to 174 nmol/l, regardless of age.

Biomarker test assays—unless otherwise indicated herein, all biomarker test assays referred to herein are performed in accordance with standard procedures employed during the 2001-2002 cycle of the National Health and Nutrition Examination Survey.

“Aromatic elixir” has the meaning ascribed to it in NF 23. Thus, an aromatic elixir will include per 1000 ml, suitable essential oils, 375 ml of syrup, 30 g of talc, and alcohol and purified water q.s. to 1000 ml. The formulation will contain at least 250 ml of alcohol, which in turn must contain between 21% and 23% C₂H₅OH.

A “child” as used herein refers to a human being having a chronological age less than 18 years. It will be understood, however, that the invention can further be limited to particular age groups, where particularly significant treatment effects can be expected. Thus, for example, any of the methods of the current invention can be performed with a child having a chronological age of less than 18 or 12 years. In the most preferred embodiments, however, the child has a chronological age of less than sixty, forty-eight, thirty-six, thirty, or twenty-four months of age

When a child is the to have a particular condition or disease, and the disease or improvement in symptoms is defined by reference to a particular diagnostic methodology, it will be understood that the child need not have undergone the particular diagnostic methodology recited, unless words to the contrary such as “actual” are employed. Rather, it will be understood that the child would have tested positive or negative under the diagnostic methodology if tested, or would have experienced the degree of improvement defined by the diagnostic methodology if tested.

### Instrument Definitions

“ABC” refers to the Aberrant Behaviors Checklist, Second Edition, by Michael G. Aman and Nirbhay N. Singh, in effect on Jan. 1, 2018 (Slosson Educational Publications, Inc., Pubs). Administration of the ABC yields a global score in addition to domain scores for irritability, social withdrawal (lethargy), stereotypic behavior, hyperactivity/noncompliance, and inappropriate speech. The methods of the present invention can be used to improve behaviors under any of the foregoing domains, or the ABC global score.

“ADOS-2” refers to The Autism Diagnostic Observation Schedule, second edition, by Catherine Lord, Susan Risi, Linda Lambrecht, Edwin H. Cook, Jr., Bennett L. Leventhal, Pamela C. DiLavore, Andrew Pickles, and Michael Rutter, in effect on Jan. 1, 2018 (Western Psychological Services, Pubs). Under the ADOS-2, the appropriate module is selected based on a participant's age and ability. Behaviors are scored in social affect and receptive repetitive behavior domains; the social affect domain is further divided into communication and reciprocal social interactions subdomains. A comparison score (or global “severity score”) is also generated of high (8-10), moderate (5-7), low (3-4), or minimal to no evidence (1 or 2). The methods of the present invention can be practiced to improve scores in any of these domains or subdomains, but are preferably practiced to improve communication and reciprocal social interaction and global severity scores.

“ASQ-3” refers to the Ages & Stages Questionnaires: A Parent-Completed Child Monitoring System, Third Edition, by Jane Squires, Elizabeth Twombly, Diane Bricker, & LaWanda Potter, in effect on Jan. 1, 2018 (Brookes Publishing, Pubs.). The instrument generates scores in the following five domains: communication, gross motor, fine motor, problem solving, and personal-social. ASQ-3 scoring under the present invention is based on the communication domain. A child is considered to have language impairment when testing under the ASQ-3 produces a score of from 1 to 3 standard deviations below the communication mean, as defined in the ASQ-3 Technical Report (Brooks Publishing 2009).

“BOSCC” refers to the Brief Observation of Social Communication Change (formerly the ADOS change) version Nov. 7, 2018, by Catherine Lord, Rebecca Grzadzinski, & So Hyun Kim (Western Psychological Services, pubs). In particular, the instrument refers to Module 1 for minimally verbal youth and toddlers (“BOSCC-T”). Administration of the instrument provides an average total score that can be used to evaluate improvements in social communication.

“DSM-5” refers to the Diagnostic and Statistical Manual of Mental Disorders, 5^{th} Edition in effect on Jan. 1, 2018 (American Psychiatric Association, Pubs). The 5^{th} edition consolidates the terms “autism” and “ASD” (autism spectrum disorder) into one umbrella diagnosis. The 5th edition also provides a severity assessment scale (levels 1-3) based on level of support needed for daily function.

“MSEL” refers to the Mullen Scales of Early Learning, AGS Edition, by Eileen M. Mullen, in effect on Jan. 1, 2018 (NCS Pearson, Inc., Pubs). Administration of the instrument yields separate Expressive Language, Receptive Language, Fine Motor, Visual Reception, and Gross Motor scores (raw scores, T-scores, and age equivalents). The instrument also yields an Early Learning Composite/Cognitive Index/Intelligence Quotient/IQ. The methods of the present invention can be practiced to yield improvements in any of the MSEL domains, using any of the aforementioned scoring systems, but they are preferably practiced to measure improvements in the expressive language domain evaluating based on raw score improvements of T-score improvements. The MSEL can also be used to define suitable childs with whom the invention may be practiced, based on the child's IQ.

“OACIS” refers to Ohio State University Clinical Impressions (Butter and Mulick, 2006; Nationwide Children's Hospital, Columbus, Ohio, USA). The OACIS is a clinical rating of severity and improvement of 10 ASD-related symptoms: social interaction, aberrant/abnormal behavior, repetitive/ritualistic behavior, verbal communication, non-verbal communication, hyperactivity/inattention, anxiety/fears, sensory sensitivities, restricted/narrow interests, autism. Each item is rated on a 7-point scale.

“PGIA” refers to Parent Global Impressions of Autism. The PGIA is a 20 item questionnaire that asks parents to rate symptoms from “none” to “extremely severe” on a 7 point Likert scale.

“PLS-5” refers to Pre-School Language Scores, Version 5, by Zimmerman, I., Steiner, V., & Pond, R. in effect on Jan. 1, 2019 (The Psychological Corporation, Pubs). Scores reported herein are preferably scaled scored based on PLS-5 normative data reported in 1-month increments for children ages 2:6-2:11.

“VABS” refers to the Vineland Adaptive Behavior Scales, Second Edition (Vineland-II), by Sparrow, S., Balla, D., & Cicchetti, D., in effect on Jan. 1, 2018 (AGS Pubs). The VABS includes domains for communication, daily living, social skills, motor skills, and adaptive behavior. The methods of the present invention can be used to improve scores under any of the foregoing domains, but are preferably used to improve communication scores.

### Discussion of Principal Embodiments

The invention can be described in terms of principal embodiments, which in turn can be recombined to make other principal embodiments and limited by sub-embodiments to make other principal embodiments.

In a first principal embodiment the invention provide a method of improving MSEL expressive communication scores in a child less than 6 years of age, wherein the child is characterized by: (a) a moderate level of autism under ADOS-2, and (b) a normal CSF folate concentration of from 39.7 nmol/l to 174 nmol/l; comprising orally administering to the child from a preservative free dropper from 5 to 25 35-60 mcl drops per day of a pharmaceutical formulation comprising a preservative-free liquid solution of 5-formyl-6(s)-tetrahydrofolate or a pharmaceutically acceptable salt thereof, at a concentration of from 20 to 30 mcg/ml, that has an absolute bioavailability of 5-formyl-6(s)-tetrahydrofolate greater than or equal to 92%.

In a second principal embodiment the invention provides a method of improving MSEL expressive communication scores in a child less than 6 years of age having a developmental or chronic cerebral nervous system disorder comprising orally administering to the child a pharmaceutical formulation comprising a therapeutically effective amount of 5-formyl-6(s)-tetrahydrofolate or a pharmaceutically acceptable salt thereof.

In a third principal embodiment the invention provides a method of improving a measure of social or intellectual functioning in a child less than 6 years of age having a developmental or chronic cerebral nervous system disorder comprising orally administering to the child a pharmaceutical formulation comprising a therapeutically effective amount of 5-formyl-6(s)-tetrahydrofolate or a pharmaceutically acceptable salt thereof, wherein the measure of intellectual functioning is selected from: (a) an MSEL cognitive index score, or an MSEL score in the expressive language, receptive language, fine motor, visual reception, or gross motor domain; (b) a BOSCC social communication score; (c) a global score under the ABC or an ABC score in the irritability, social withdrawal, stereotypic behavior, hyperactivity, or inappropriate speech domain; (d) a global severity score under the ADOS-2 or a reciprocal social interaction or communication sub-score under the ASOS-2; or (e) a VABS total score or a VABS score in the communication, daily living, social skills, motor skills, or adaptive behavior domain.

In a fourth principal embodiment the invention provides a method of treating a developmental or chronic cerebral nervous system disorder in a child comprising orally administering to the child a pharmaceutical formulation comprising an aqueous solution when ingested of a therapeutically effective amount of 5-formyl-6(s)-tetrahydrofolate or a pharmaceutically acceptable salt thereof.

In a fifth principal embodiment the invention provides a method of treating or preventing a developmental or chronic cerebral nervous system disorder in the unborn child of a maternal human in need thereof, comprising orally administering to the maternal human a pharmaceutical formulation comprising a therapeutically effective amount of 5-formyl-6(s)-tetrahydrofolate or a pharmaceutically acceptable salt thereof. In a particularly preferred subembodiment, the method prevents a neural tube defect.

In a sixth principal embodiment the invention provides a drug product comprising a multi-dose dropper comprising from 5 to 50 ml of an aqueous oral solution of 5-formyl-(6S)-tetrahydrofolate or a pharmaceutically acceptable salt thereof, wherein the concentration of 5-formyl-(6S)-tetrahydrofolate in the solution is from 20 to 30 mg/ml based on the weight of the anhydrous free acid, and each drop dispensed from the dropper comprises from 35 to 150 microliters of solution.

### Discussion of Subembodiments

The invention can further be understood with reference to various subembodiments which can modify any of the principal embodiments. It will be understood that these subembodiments can be combined in any manner that is both mathematically and physically possible to create additional subembodiments, which in turn can modify any of the principal embodiments. It will also be understood that any of the features of the methods of the present invention apply equally to the devices of the present invention, and vice versa. However, certain verbiage can be employed in the description of the devices of the present invention, which is more appropriate when defining a device.

### Conditions and Disorders Treatable by the Present Invention

In any of the foregoing embodiment, the child can be defined based on the developmental or chronic cerebral nervous system disorder from which the child suffers. Thus, in some subembodiments the child has autism based upon DSM-5 criteria, optionally characterized as severity level 1, 2, or 3. In other subembodiments the child has language impairment corresponding to from 1 to 3, 4, or 5 standard deviations below the mean on ASQ-3 for communication. In other subembodiments the child has language impairment corresponding to a scaled score ≤90 or 85 or 80 based on PLS-5 normative data reported in 1-month increments for children ages 2:6-2:11. In still further subembodiments the child has intellectual impairment corresponding to a composite standard score on the MSEL of ≤90 or 85 or 80. In other subembodiments the child has language impairment corresponding to an expressive language standard score on the MSEL of ≤90 or 85 or 80. In still other subembodiments the child has epilepsy defined by abnormal EEG activity, anti-epileptic medication, or a history of seizures. In further subembodiments the child has a moderate level of autism symptoms on ADOS-2.

In one particular subembodiment the child has autism based on DSM-5 criteria, language impairment corresponding to from 1 to 3 standard deviations below the mean on ASQ-3 for communication, and a moderate level of autism symptoms on ADOS-2. In another particular subembodiment the child has autism based on DSM-5 criteria and epilepsy as defined by abnormal EEG activity, anti-epileptic medication, or a history of seizures.

In still further subembodiments, the child has a developmental or chronic cerebral nervous system disorder selected from a learning disorder, a memory disorder, a communication disorder, a seizure disorder, epilepsy, depression, schizophrenia, and autism. In another subembodiment, the developmental or chronic cerebral nervous disorder is a neurodegenerative disease or a neuropsychiatric disease.

In another subembodiment the methods of the present invention are practiced in a child in whom the developmental or chronic cerebral nervous system disorder is associated with imbalanced, perturbed, or impaired tetrahydrofolate utilization in a tissue selected from the cerebellum, cortex, placenta, embryo, or ovary, and the method preferentially increases concentrations of tetrahydrofolate in one or more of the tissues compared to another folate selected from leucovorin, L,5-MTHF, and folic acid.

In another subembodiment the methods of the present invention are practiced in a child in whom the developmental or chronic cerebral nervous system disorder is associated with impaired neurogenesis selected from neuronal synthesis, neuronal integration, neuronal differentiation, abnormal axonal branching, impaired myelin synthesis, disturbed cortical activity, and impaired neuronal connectivity, and the method improves the neurogenesis.

For the purposes of this invention, epilepsy includes both general and partial epilepsy as well as paroxysmal EEG epileptiform abnormalities. In one subembodiment, the child has a history of seizures within the previous 6 months. In another subembodiment, the child has had at least one seizure in the previous 6 months and the administration of 5-formyl-(6S)-tetrahydrofolate prevents or reduces the seizures. In still another preferred embodiment, the child has had at least one seizure in the previous 6 months, is taking anti-seizure medication for the seizures, and the administration of 5-formyl-(6S)-tetrahydrofolate prevents or reduces the seizures.

In various subembodiments, the methods of the current invention are practiced based on the antibody status of the child, i.e. antibody concentration and/or antibody type. In some subembodiments the child is negative for the blocking folate receptor auto-antibody, the binding folate receptor auto-antibody, or both. In other subembodiments the child is positive for the blocking folate receptor auto-antibody, the binding folate receptor auto-antibody, or both.

In still further subembodiments, the child is defined by the concomitant medications he or she is taking. The child treated by the methods of the present invention preferably is not receiving any concomitant anti-psychotic medications during treatment with the 5-formyl-(6S)-tetrahydrofolate, although in one subembodiment the child is on aripiprazole or risperidone or a pharmaceutically acceptable salt thereof. In one subembodiment, the child is on aripiprazole or risperidone or a pharmaceutically acceptable salt thereof when treatment with the 5-formyl-(6S)-tetrahydrofolate begins. This subembodiment preferably comprises commencing the 5-formyl-(6S)-tetrahydrofolate at a dose equal to ½ the normal dose, monitoring the child for adverse events, and increasing the dose after a suitable period without observing any adverse events. In another subembodiment the child is on aripiprazole or risperidone or a pharmaceutically acceptable salt thereof when treatment with the 5-formyl-(6S)-tetrahydrofolate begins, and the method further comprises, after a period of steady state behavior, removing the aripiprazole or risperidone from the therapy.

Anti-epileptic medications with which the invention can be practiced include, for example, acetazolamide, brivaracetam, carbamazepine, clobazam, clonazepam, eslicarbazepine acetate, ethosuximide, ezogabine, gabapentin, lacosamide, lamotrigine, levetiracetam, nitrazepam, oxcarbazepine, perampanel, piracetam, phenobarbital, phenytoin, pregabalin, primidone, retigabine, rufinamide, sodium valproate, stiripentol, tiagabine, topiramate, valproate, vigabatrin, and zonisamide, and their pharmaceutically acceptable salts. Once again, this subembodiment preferably comprises commencing the 5-formyl-(6S)-tetrahydrofolate at a dose equal to ½ the normal dose, monitoring the child for adverse events, and increasing the dose after a suitable period without observing any adverse events. In another subembodiment the child is on anti-epileptic medication when treatment with the 5-formyl-(6S)-tetrahydrofolate begins, and the method further comprises, after a period of steady state behavior, removing the anti-epileptic medication from the therapy.

In an alternative subembodiment, the development or chronic cerebral nervous system disorder is communication impairment. Communication impairment can be defined in a variety of ways, but in some subembodiments is defined as (a) an age of from 3 to 14 years and less than 25 functional words; or (b) a failure on the Red Flags for Toddlers in ASD test for children from 16 to 24 months old. In yet another subembodiment, the child has a communication score on the Vineland Adaptive Behavior Scale of less than 70, 69, 68, 67, 66, or 65.

The methods can also be practiced regardless of the intelligence quotient (IQ) of the child being treated, although they are preferably practiced in children with below average IQ levels. In particular embodiments, the methods are practiced in a child having a Cognitive Score of less than 90, 85, 80, 75, 70, 65, or 60 and/or greater than 40 or 55 as measured by the MSEL.

In any of the principal embodiments or subembodiments of the present invention, the child or maternal human preferably has normal folate concentrations in the cerebral spinal fluid, as described in the definitions section of this document.

### Definition of “Clinically Meaningful Improvement”

An improvement in symptoms as described herein can be a numeric improvement or it can represent a clinically meaningful change or clinically meaningful improvement. When an improvement represents clinically meaningful change or improvement, it will be understood that the change or improvement corresponds to a degree of improvement observed when evaluated using CGI and/or CaGI anchors as discussed herein. The CGI and CaGI anchors are included as prospective and retrospective assessments of the participant child's global ASD verbal communication and language ability in a clinical trial at each assessment and retrospectively at the end of treatment. For the static, prospective measure, Caregivers and Clinicians independently rate the patient's level of severity (global and language, separately) on a 6-point verbal rating scale ranging from “none” (0) to “very severe” (5) at each assessment. Caregivers and clinicians also use a retrospective assessment of improvement over the course of treatment to rate the participant child's improvement (global and language, separately) on a 7-point verbal rating scale ranging from “very much improved” (1) to “very much worse” (7).

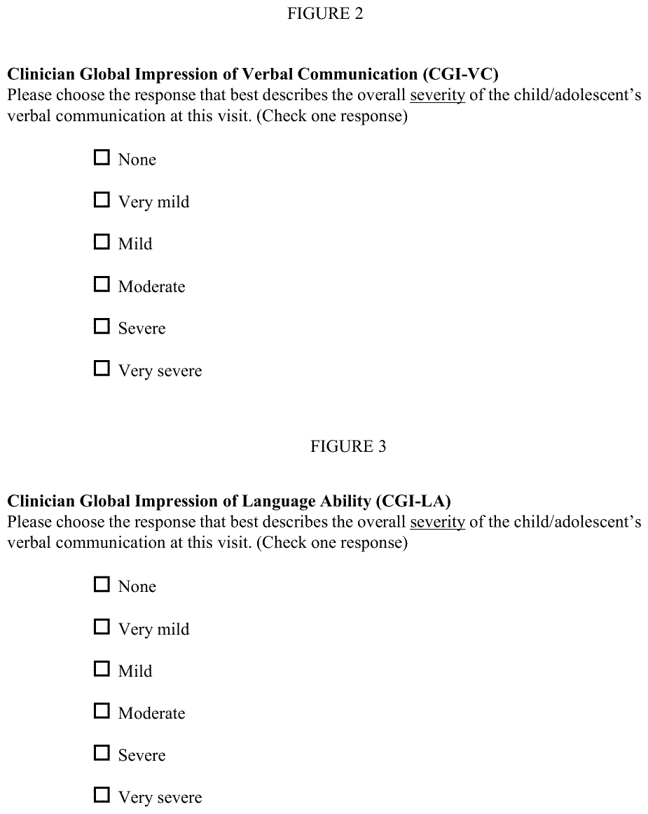

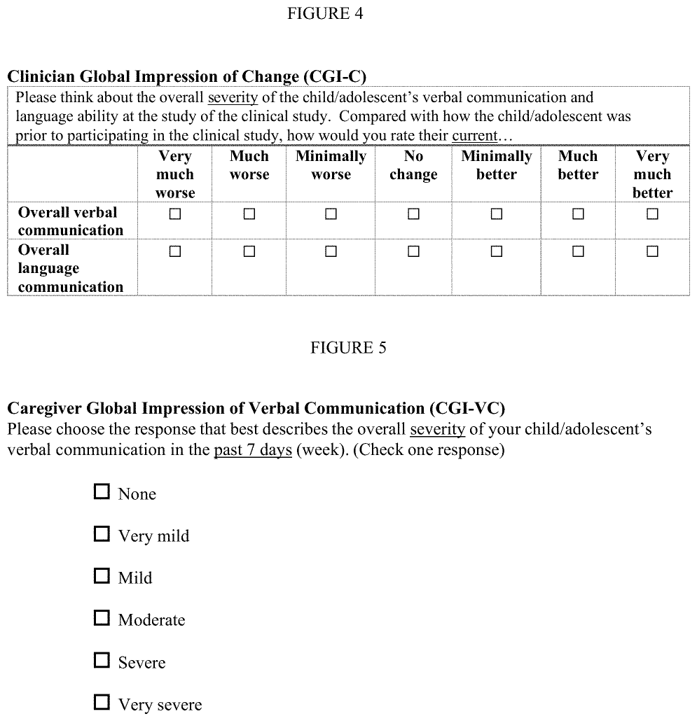

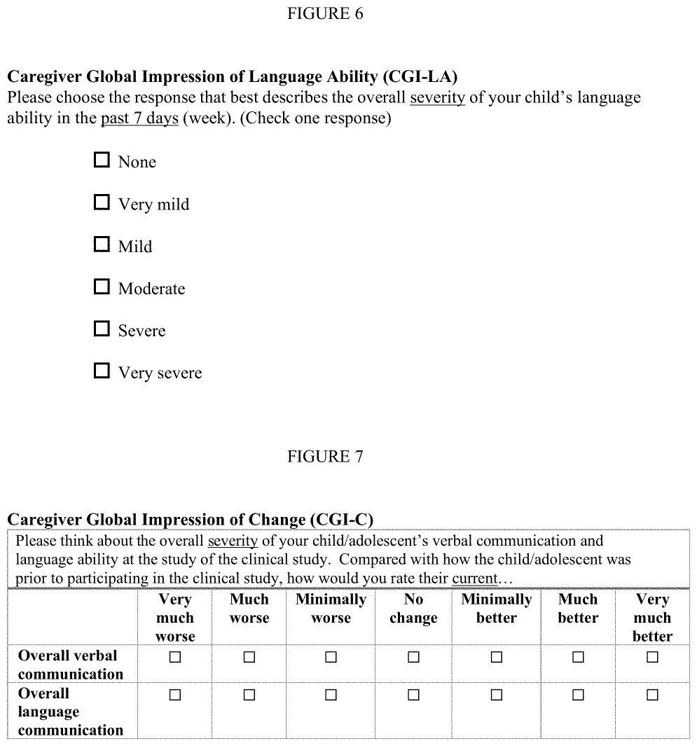

Exemplary CGI and CaGI Anchors are included in FIGS. 2-7 hereto. In addition, one could use as an anchor any of the severity scores included in the OACIS or PGIA. Unless otherwise specified, the CGI or CaGI will query “language ability,” “verbal communication,” or “expressive language/speech,” particularly when coupled with the MSEL expressive language domain.

Anchor based methods are carried out using treatment-agnostic, blinded clinical trial data to understand the parameters and thresholds for diagnosing meaningful change on the MSEL or other clinical outcome assessment. Correlations between the clinician and caregiver prospective and retrospective anchors and the MSEL are evaluated with a value of >0.35; with anchors meeting these criteria carried forward in the anchor based analysis. Correlation coefficients are assessed between the MSEL and the anchors raw and change scores between Baseline (Day 1) and the end of the study (preferably Week 12).

To determine the anchor-based meaningful change, subjects are grouped by change on the MSEL on qualifying anchors. Using the CGI and CaGI as anchors, subjects are classified into response groups depending upon their level of change over the course of the study. For each anchor, change within each anchor group, in terms of improvement levels from baseline are calculated on the BOSCC. The n, mean change, median change, standard deviation (SD), confidence intervals and standardized effect size (SES) are reported for each level of change on each anchor.

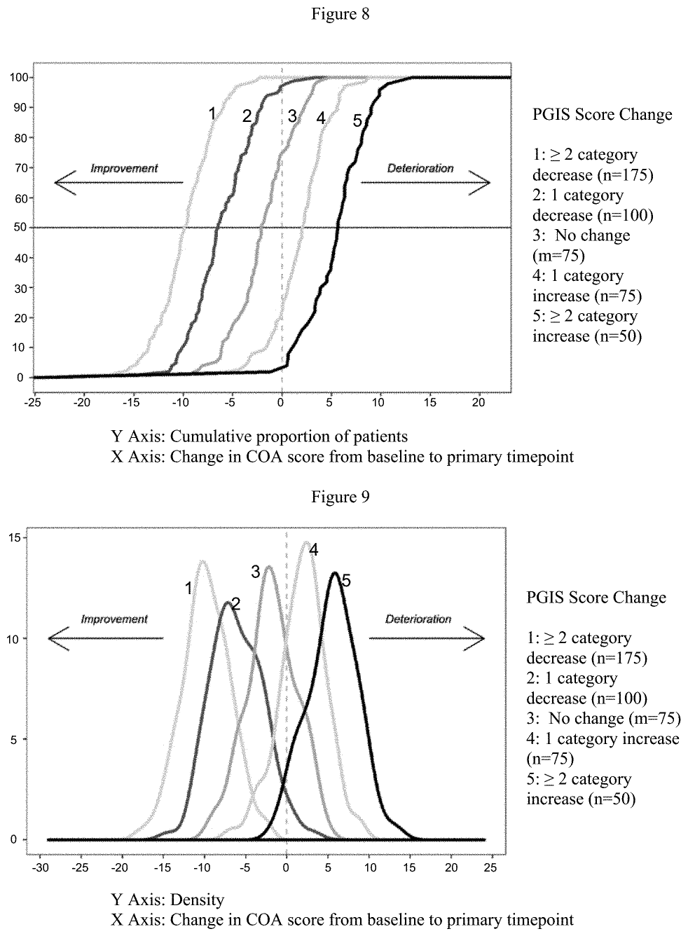

To illustrate this relationship, Cumulative Distribution Function “(CDF”) and Kernel Density Plots (“KDP”) are presented for each anchor and evaluated and calculated as cumulative change in MSEL expressive language domain scores for all available changes from baseline (Day 1) to the end of the study. Absolute change from Baseline (Day 1) MSEL expressive domain scores are expressed on the x-axis and the cumulative number of subjects who have a given score change for CDF curves and density function for kernel plots are presented on the y-axis. Exemplary CDF and KDP graphs are depicted in FIGS. 8 and 9.

Unless stated to the contrary herein, a clinically meaningful improvement is achieved when a statistically significant change on the clinical outcome assessment (p<0.05) corresponding to a change in at least one, two or three severity levels on a six point CGI or CaGI severity rating scale is observed. Thus, a clinically meaningful improvement for MSEL expressive communication is achieved when a statistically significant change on the expressive communication domain (p<0.05) corresponding to a change in at least one, two or three severity levels on the CGI-S anchor (verbal communication) is observed.

### Dosing

In various subembodiments, the therapeutically effective amount of 5-formyl-(6S)-tetrahydrofolate comprises from 0.01 to 5.0 mg/kg/day, from about 0.15 to about 2 or 2.5 mg/kg/day, from about 0.25 to about 1.5 or 2.0 mg/kg/day, or from about 0.5 or 1 mg/kg/day. Alternatively, the dose can be from about 0.1 to about 0.5 mg/kg/day, from about 0.5 to about 1.0 mg/kg/day, from about 1.0 to about 1.5 mg/kg/day, from about 1.5 to about 2.0 mg/kg/day, or from about 2.0 to about 2.5 mg/kg/day. Alternatively, the dose can be about 0.5, about 1.0, or about 1.5 mg/kg/day. A preferred dosing frequency is twice per day. The foregoing doses are based on the weight of the free acid of the anhydrous 5-formyl-(6S)-tetrahydrofolate.

In this context, the term “about” is used because of the necessity of manufacturing fixed strength dosage forms, and the inability to correlate the fixed strength precisely with the mg/kg dose for a variety of body weights. All doses are preferably administered in divided doses two or three times daily, preferably not to exceed 50 or 25 or 12.5 or 10.0 or 7.5 or 5 mg/day.

In one subembodiment the therapeutically effective amount comprises from about 0.01 to about 2.0 mg/kg/day, up to a maximum of 50 mg/day, based on the weight of the anhydrous free acid of the 5-formyl-(6S)-tetrahydrofolate. In another subembodiment the therapeutically effective amount comprises from about 0.1 to about 1.0 mg/kg/day, up to a maximum of 25 mg/day, based on the weight of the anhydrous free acid of the 5-formyl-(6S)-tetrahydrofolate. In yet another subembodiment the therapeutically effective amount comprises from about 0.25 to about 0.5 mg/kg/day, up to a maximum of 15 mg/day, based on the weight of the anhydrous free acid of the 5-formyl-(6S)-tetrahydrofolate.

In still another subembodiment the therapeutically effective amount comprises from about 5 to about 25 mg/day, based on the weight of the anhydrous free acid of the 5-formyl-(6S)-tetrahydrofolate. In another subembodiment the therapeutically effective amount comprises from about 5 to about 10 mg/day, based on the weight of the anhydrous free acid of the 5-formyl-(6S)-tetrahydrofolate.

In other subembodiments the therapeutically effective amount comprises about 0.4, 0.5, 0.8, 1.0, 2.5, 5.0, 7.5, 10.0, 12.5, 15.0, 17.5, 20.0, 22.5, 25.0, 27.5, 30.0, 32.5, 35.0, 37.5, 40.0, 42.5, 45.0, 47.5, or 50.0 mg/day, or any range between any of the foregoing amounts, based on the weight of the anhydrous free acid of the 5-formyl-(6S)-tetrahydrofolate.

In one subembodiment the invention comprises, after from 2 to 4 weeks, evaluating the child for negative side effects from the 5-formyl-(6S)-tetrahydrofolate therapy and, if the child is not suffering the negative side effects, doubling the dose of the 5-formyl-(6S)-tetrahydrofolate. For example, the therapeutically effective amount may comprise a dose of from about 0.25 to about 2 mg/kg/day after 2 to 4 weeks of the therapeutically effective period of time, and half of the dose for the first 2 to 4 weeks.

Conversely, the invention can be practiced by reducing the dose if adverse events are observed. Thus, in still another subembodiment, the methods further comprise, after a suitable observation period, preferably from 2 to 4 weeks, evaluating the child for negative side effects from the 5-formyl-(6S)-tetrahydrofolate administration and, if the child is suffering negative side effects, reducing (preferably by ½) the initial dose (e.g. from 0.25 to 2 mg/kg/day) of the 5-formyl-(6S)-tetrahydrofolate.

In the prevention of neural tube defects, the methods of the present invention comprise in some subembodiments administering to the mother of the child during pregnancy from 0.4 to 25.0 mg/day of 5-formyl-(6S)-tetrahydrofolate or a pharmaceutically acceptable salt thereof based on the weight of the anhydrous free acid. Alternatively, the methods of the present invention comprise administering to the mother of the child during pregnancy from 0.8 to 2.5 mg/day of 5-formyl-(6S)-tetrahydrofolate or a pharmaceutically acceptable salt thereof based on the weight of the anhydrous free acid.

### Pharmaceutical Dosage Forms

Pharmaceutical compositions for practicing the methods of the invention are further provided comprising a therapeutically effective amount of a 5-formyl-(6S)-tetrahydrofolate, or a pharmaceutically acceptable salt thereof, and one or more pharmaceutically acceptable excipients. In any of the embodiments of the present invention, the 5-formyl-(6S)-tetrahydrofolate can be administered as the calcium salt. Furthermore, the 5-formyl-(6S)-tetrahydrofolate can be administered as the pentahydrate calcium salt.

A “pharmaceutically acceptable” excipient is one that is not biologically or otherwise undesirable, i.e., the material can be administered to a child without causing any undesirable biological effects or interacting in a deleterious manner with any of the other components of the pharmaceutical composition in which it is contained. The carrier can be selected to minimize any degradation of the active ingredient and to minimize any adverse side effects in the child, as would be well known to one of skill in the art. The carrier can be a solid, a liquid, or both.

In one particularly preferred embodiment, when the drug product is administered as an oral liquid, as a formulation that is free of preservatives. Other excipients which should be avoided regardless of dosage form include lactose and dyes. A particularly preferred excipient for stabilizing the formulation, particular when the formulation is a liquid solution, is sodium gluconate, and the sodium gluconate is preferably present in a stabilizing effective amount.

The 5-formyl-(6S)-tetrahydrofolate is preferably administered orally (including both traditional oral administration as well as buccal and sub-lingual). Suitable dosage forms include tablets (including buccal tablets and orally dissolving tablets), film strips, capsules, powders (granulates, beads, particles, etc.), and liquids including drops and syrups, solutions and suspensions. In one particular subembodiment the 5-formyl-(6S)-tetrahydrofolate is present as a liquid in a kit along with a metered pipette for delivering a carefully measured dose based on the child's or woman's weight.

In one particular embodiment, the 5-formyl-(6S)-tetrahydrofolate is administered as individual drops from a multi-dose dropper bottle in a preservative free liquid solution. The concentration of 5-formyl-(6S)-tetrahydrofolate in the solution will typically range from about 10 to about 50 mg/ml or about 15 to about 35 mg/ml, based on the weight of the anhydrous free acid, and each drop will typically comprise from about 30 to about 150 microliters or from about 35 to about 60 microliters of solution.

In one particularly preferred embodiment, the 5-formyl-(6S)-tetrahydrofolate is administered dropwise in the form of an oral solution, the concentration of 5-formyl-(6S)-tetrahydrofolate in the solution is about 25 mg/ml based on the weight of the anhydrous free acid, and the volume of each drop is about 40 microliters.

Suitable carriers and their formulations are described in Remington: The Science and Practice of Pharmacy (19th ed.) ed. A. R. Gennaro, Mack Publishing Company, Easton, Pa., 1995. Oral administration of a solid dose form can be, for example, presented in discrete units, such as hard or soft capsules, pills, cachets, lozenges, or tablets, each containing a predetermined amount of at least one of the disclosed compound or compositions. In some forms, the oral administration can be in a powder or granule form. In some forms, the oral dose form is sub-lingual, such as, for example, a lozenge. In such solid dosage forms, the compounds of the present invention are ordinarily combined with one or more adjuvants. Such capsules or tablets can contain a controlled-release formulation. In the case of capsules, tablets, and pills, the dosage forms also can comprise buffering agents or can be prepared with enteric coatings.

### Preferred PK/Dosage Form Combinations

In one series of preferred subembodiments, the dosage form is selected so that when it is swallowed or ingested it is an aqueous solution. Thus, the dosage form can be an aqueous solution, or it can be a film strip dosage form or orally dissolving tablet that dissolves in the mouth completely before swallowing. Thus, in one subembodiment, the pharmaceutical formulation comprises an aqueous solution when ingested of a therapeutically effective amount of 5-formyl-6(s)-tetrahydrofolate or a pharmaceutically acceptable salt thereof.

In some subembodiments, the formulation is an aqueous solution free of aromatic elixir at a pH of 7.8±1.0. In other subembodiments, the formulation is an alcohol-free aqueous solution at a pH of 7.8±1.0. In other subembodiments, the formulation is an alcohol-free preservative-free aqueous solution at a pH of 7.8±1.0. By “preservative-free” is meant that the solution is also not “self-preserved” as that term is commonly understood.

In still other subembodiments, (a) the formulation is an alcohol-free preservative-free aqueous solution at a pH of 7.8±1.0; and (b) the formulation comprises 5-formyl-6(S)-tetrahydrofolate or a pharmaceutically acceptable salt thereof in a concentration of from 20 to 30 mg/ml. In yet another subembodiment (a) the formulation is an alcohol-free preservative-free aqueous solution at a pH of 7.8±1.0; and (b) the formulation consists essentially of water, 5-formyl-6(s)-tetrahydrofolate or a pharmaceutically acceptable salt thereof in a concentration of from 20 to 30 mg/ml, sodium gluconate, and optionally a pH adjusting agent such as sodium hydroxide of hydrochloric acid..

In still further subembodiments the formulation is provided as a film strip dosage form. For example, the formulation is provided as an ethanol-free film strip dosage form comprising polyethylene glycol, polyvinyl alcohol, and rice starch.

Other subembodiments are defined by the improved pharmacokinetics achieved when 5-formyl-6(S)-tetrahydrofolate is ingested as an aqueous solution. Thus, in one subembodiment, the formulation when orally administered preferentially increases concentrations of tetrahydrofolate compared to 5-formyl-6(R,S)-tetrahydrofolate.

In another subembodiment the formulation comprising 12.5 mg of 5-formyl-6(S)-tetrahydrofolate calcium salt has been shown to produce a pharmacokinetic response selected from: (a) an absolute bioavailability of total reduced l-folates exceeding 92%, 93%, 94%, 95%, or 96%; (b) a cₘₐₓ of total reduced l-folates greater than 410 ng/ml, 420 ng/ml, 440 ng/ml, or 460 ng/ml; (c) a cₘₐₓ of 1,5-methyl-THF greater than 385 ng/ml, 400 ng/ml, 415 ng/ml, or 430 ng/ml; (d) a cₘₐₓ of 5-Formyl-THF less than 40 ng/ml, 30 ng/ml, 20 ng/ml, or 10 ng/ml; (e) a ratio of circulating 5-methyl-THF to 5-formyl-THF in excess of 10:1, 20:1, 50:1, 75:1, or 100:1; or (f) a combination thereof. In a particularly preferred embodiment, each of the foregoing pharmacokinetics is observed on a statistically significant basis (p<0.05) to be superior to the comparable pharmacokinetic measure observed for the liquid elixir studied in McGuire 1988.

All of the pharmacokinetics measurements recited above are performed according to the methods described in McGuire 1988 unless otherwise stated herein, measuring l-casei activity as a surrogate for total reduced folate concentrations and Strep. faecalis activity as a surrogate for 5-formyl-THF. As further reported by McGuire 1988, the testing is preferably performed in healthy adults in the fasting state. Any further details needed to evaluate the foregoing pharmacokinetic parameters can be determined with reference to U.S. FDA's Population Pharmacokinetics Guidance.

A “preservative” refers to an inactive excipient in the formulation in sufficient amounts to ensure that the formulation remains free of microbiological contaminants when stored 5 degrees celsius over a two year period, according to standards and guidances published by the US Food and Drug Administration.

### Drug/Device Combinations

In one particular embodiment the drug is administered using a preservative-free multi-dose dropper, which allows for maintenance of sterility when multiple doses of a preservative-free solution are administered. Thus, in another principal embodiment the invention provides a drug product comprising a multi-dose dropper comprising from 5 to 20 ml of an aqueous liquid oral solution of 5-formyl-(6S)-tetrahydrofolate or a pharmaceutically acceptable salt thereof. In a particularly preferred embodiment the oral solution is sterile and preservative free, and the dropper restricts the ingress of contaminants from the surrounding air to the solution during use, as through a silicon membrane, and further restricts the return or regress of the liquid solution back into the nozzle of the dropper after dosing is complete. By “sterile” is meant that the solution is filled in an aseptic environment that minimizes the potential for microbial contamination. Exemplary dropper designs are described, for example, in U.S. Pat. Nos. 8,517,222 B1, 8,794,490 B1, 8,827,124 B1, 8,863,998 B1, 8,986,266 B1, and U.S. Patent Publication Nos. US 2011/0155770 A1, US 2014/0231536 A1, and US 2017/0029175 A1 to Nemera SA.

The drug device combination can dispense any of the solutions mentioned elsewhere in this document. However, in one particular embodiment the solution in the drug product comprises from 20 to 30 mg/ml of 5-formyl-(6S)-tetrahydrofolate based on the weight of the anhydrous free acid, and the dropper dispenses from 35 to 60 microliters per drop. In one particular embodiment the solution comprises a stabilizing effective amount of sodium gluconate. A preferred liquid solution comprises approximately 40 grams of sodium gluconate and approximately 25 grams of levoleucovorin calcium (based on the weight of the anhydrous free acid) in one liter of water, at a pH of approximately 7.8.

As mentioned previously, a multi-dose dropper is particularly advantageous because it allows for a single drug product to be used multiple times in the methods of the present invention. Thus, in any of the methods of the current invention, the drug product described herein is used to administer the 5-formyl-(6S)-tetrahydrofolate multiple times, i.e. on 5, 10, 15 or even more dosing events.

As noted above, in a sixth principal embodiment the invention provides a drug product comprising a multi-dose dropper comprising from 5 to 50 ml of an aqueous oral solution of 5-formyl-(6S)-tetrahydrofolate or a pharmaceutically acceptable salt thereof, wherein the concentration of 5-formyl-(6S)-tetrahydrofolate in the solution is from 20 to 30 mg/ml based on the weight of the anhydrous free acid, and each drop dispensed from the dropper comprises from 35 to 150 microliters of solution.

In one subembodiment, the oral solution in the drug product is sterile and preservative free. In another subembodiment, the dropper restricts the ingress of contaminants from the surrounding air to the solution during use and the regress of the liquid solution that has been dispensed from the dropper. In yet another subembodiment, the solution in the dropper comprises from 20 to 30 mg/ml of 5-formyl-(6S)-tetrahydrofolate based on the weight of the anhydrous free acid, and the dropper dispenses from 35 to 60 microliters per drop. In still another subembodiment the concentration of 5-formyl-(6S)-tetrahydrofolate in the solution is about 25 mg/ml based on the weight of the anhydrous free acid, and the dropper dispenses about 40 microliters per drop. In yet another subembodiment the solution consists essentially of water, 5-formyl-(6S)-tetrahydrofolate calcium, sodium gluconate, and optionally a pH adjusting agent such as sodium hydroxide or hydrochloric acid, at a pH of from 6.8 to 8.8.

Importantly, in any of the methods of this invention, the 5-formyl-(6S)-tetrahydrofolate or pharmaceutically acceptable salt thereof is preferably administered from the preservative free dropper described herein, as the liquid solution described herein.

### Miscellaneous Subembodiments

The methods can also be practiced by controlling the diet of the child. In a particularly preferred embodiment, all dairy products are restricted, and the administration of the 5-formyl-(6S)-tetrahydrofolate occurs in the substantial absence of dairy products, such as cheese, milk or butter from cows, sheep, goats, or camels.

The methods can also be practiced by the concomitant administration of vitamin supplements, even vitamin supplements in excess of the recommended daily allowance. Thus, in another subembodiment the methods of the present invention further comprise administration of supplemental vitamin B12.

Folic acid, however, is preferably omitted from the child's diet. Thus, in another embodiment the administration of 5-formyl-(6S)-tetrahydrofolate occurs in the substantial absence of folic acid supplements, and preferably occurs in the substantial absence of all folic acid, including folic acid derived from fortified grains. In another embodiment the methods comprise reducing concentrations of unmetabolized folic acid in the child.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

### Example 1. Patterns of Folate Absorption Following High Dose Oral Administration of Different Folates

For this study, 4 mg/kg folate was administered as a single dose to 250-300 g male Wistar rats. Folates studied were folic acid (“PGA”), 5-methyl-(6S)-THF, folinic acid, and 5-formyl-(6S)-tetrahydrofolate. For each form of folate 4 time points of 30 min, 1 h, 2 h and 4 h were evaluated. For each time point 2 rats were used. The folate form dissolved in 0.5 ml buffered saline was administered orally using a blunt needle attached to a 1 ml syringe. The rats were euthanized by CO2 inhalation.

Blood was drawn cardiac puncture, allowed to clot for 30 min, serum separated, diluted in equal volume of 4% ascorbate pH 7.2 and kept frozen at −20 C. Folate forms in the serum was determined by HPLC and the peaks were quantified by comparing to known standards as described below.

Folate standards (5-MTHF, FA, THF and Formyl-THF) and 13C-labeled stable isotope internal standards (13C5-5-MTHF, 13C5-FA and 13C-Formyl-THF) were obtained from Schircks Laboratories (Switzerland). Formic acid, ascorbic acid and dithiothreitol (DTT) were obtained from Fluka and Optima LC-MS grade methanol from Fisher Scientific. Stock standards for each folate metabolite and internal standards were prepared as 1 mmol/L solutions in water containing 2 mg/mL ascorbic acid and stored at −80° C. Microtiter plates used for were purchased from NUNC.

Liquid chromatography electrospray ionization mass spectrometry (LC-ESI-MS/MS) analysis of plasma 5-MTHF, FA, THF, Formyl-THF was performed on a Sciex 5500QTRAP mass spectrometer (Foster City, Calif., USA) coupled with a Shimadzu ultra-high pressure Nexera chromatograph system (Kyoto, Japan). The MS/MS experiments were performed under positive electrospray ionization (+ESI) with multiple-reaction monitoring (MRM) using a Turbolon Spray electrospray source operating at a voltage of 5.5 kV and desolvation temperature of 500° C. The selected precursor and fragment ions used for the measurement of unlabeled and labeled folates are summarized in Table 1. The calibration curve was prepared in water containing 2 mg/mL ascorbic acid over a range of 12.5-400 nmol/L for each folate analyte.

Sample preparation involved combining 50 μl of blank, standard, control or sample with 10 μl 62.5 mM dithiothreitol, 20 mg/ml ascorbic acid and 2.5 μM 13C5-5-MTHF, 13C5-FA, 13C-Formyl-THF in 1.5 mL Eppendorf tubes, mixed by vortex and incubated for 10 minutes at room temperature in the dark. Next, samples were deproteinized with 5 volumes of methanol containing 2 mg/ml of ascorbic acid and vortexed and centrifuged at 14800 rpm for 10 minutes. Supernatant was loaded in a 96-well microtiter plate for analysis. The samples were analyzed following injection of 10 μL of extract on a Synergi Hydro 4μ 150×3 mm with a 4×2 mm SecurityGuard column (Phenomenex, CA, USA) maintained at 40° C. (Phenomenex) and eluted in a gradient with buffer A (100% water with 0.1% formic acid) and buffer B (100% Methanol with 0.1% formic acid). The flow rate was 0.5 mL/min, with a gradient over a total run time of 6 min: 0.0-2.0 min, 0-100% B; 2.5 min, 100% B; 2.6-6.0 min, 0% B.

Total analytical analysis, including column re-equilibration, was 6 minutes. Eluent flow from the column was diverted to waste at the beginning and end of each run and was only directed to the source for the period from 1.5-3.5 minutes. The LC-MS/MS data was acquired and processed using Analyst 1.5.2 software (Sciex).

Folate absorption patterns following administration of four different types of folates are given in Tables 1-4. Values reported include both D- and L-forms.

TABLE 1a
Folic Acid Administration

| | 5-MTHF | Folic Acid | THF | 5-FoTHF | Total Uptake |
|---|---|---|---|---|---|
| | (ng/mL) | (ng/mL) | (ng/mL) | (ng/mL) | (ng/mL) |
| 0.5 hr | 72.8 | 42.4 | 10.7 | | 126.0 |
| 1 hr | 112.9 | 235.5 | 5.9 | | 354.3 |
| 2 hr | 101.4 | 134.0 | 7.9 | | 243.3 |
| 4 hr | 109.7 | 120.6 | 22.4 | | 252.7 |

TABLE 1b
5-methyl-(6S)-THF Administration

| | 5-MTHF | Folic Acid | THF | 5-FoTHF | Total Uptake |
|---|---|---|---|---|---|
| | (ng/mL) | (ng/mL) | (ng/mL) | (ng/mL) | (ng/mL) |
| 0.5 hr | 184.1 | | 4.4 | | 188.5 |
| 1 hr | 214.8 | | 9.0 | | 223.8 |
| 2 hr | 249.7 | | 8.7 | | 258.4 |
| 4 hr | 191.4 | | 19.6 | 5.1 | 216.2 |

TABLE 1c
Folinic acid Administration

| | 5-MTHF | Folic Acid | THF | 5-FoTHF | Total Uptake |
|---|---|---|---|---|---|
| | (ng/mL) | (ng/mL) | (ng/mL) | (ng/mL) | (ng/mL) |
| 0.5 hr | 109.7 | | 5.5 | 80.1 | 195.3 |
| 1 hr | 115.7 | | 5.9 | 55.9 | 177.4 |
| 2 hr | 103.3 | | 5.1 | 27.5 | 135.9 |
| 4 hr | 120.7 | | 7.2 | 21.7 | 149.6 |

TABLE 1d
5-formyl-(6S)-tetrahydrofolate Administration

| | 5-MTHF | Folic Acid | THF | 5-FoTHF | Total Uptake |
|---|---|---|---|---|---|
| | (ng/mL) | (ng/mL) | (ng/mL) | (ng/mL) | (ng/mL) |
| 0.5 hr | 161.6 | | 22.9 | 68.2 | 252.6 |
| 1 hr | 241.9 | | 35.8 | 97.3 | 375.0 |
| 2 hr | 144.1 | | 9.0 | 19.7 | 162.9 |
| 4 hr | 173.5 | | 39.1 | 7.0 | 216.1 |

### Observations:

1. Much better THF and 5-MTHF are obtained for 5-formyl-6S-THF compared to folinic acid, especially at one hour, which is the most representative timepoint for evaluating folate absorption because it occurs before folates can disappear from serum through tissue distribution in the organism.

2. One would expect THF concentrations from the administration of folinic acid to be only half the THF concentration for an equal dose of 5-formyl-6S-THF, since folinic acid only comprises 50% of the natural L-isomer. However, in these tests folinic acid generates a concentration of THF that is less than one fifth the concentration of THF from an equal dose of 5-formyl-6S-THF, suggesting significant interference with THF metabolic pathways.

3. The unexpected superiority of 5-formyl-6S-THF is also proven by a comparison to 5-methyl-(6S)-THF. THF generation and total uptake from 5-formyl-6S-THF administration is unexpectedly much greater than seen with 5-methyl-(6S)-THF administration.

### Example 2: Tissue Distribution of Folates 24 h Post Oral Administration of Various Folate Forms

This study evaluated the difference in folate absorption in non-pregnant rats, pregnant rats, pregnant rats injected with a control antibody, and pregnant rats injected with antibody to the folate receptor alpha. Eight (8) rats were assigned to each group for a total of thirty-two (32) rats. Two rats within each group were administered a single oral dose of PGA, 5-methyl-(6S)-THF, folinic acid or 5-formyl-(6S)-THF as a liquid solution and sacrificed at 24-hours to evaluate folate absorption patterns. The total amount of each form of folate administered to each rat was 4 mg/kg.

Serum was withdrawn immediately before the rat was sacrificed. For extraction of folate from serum, an aliquot of the serum was diluted in 3 volumes of phosphate buffer pH 5.5, placed in a boiling water bath for 10 min, cooled, the protein precipitate separated by centrifugation and the clear supernatant was assayed for folate concentration. Serum concentrations of total 5-MTHF (D- and L-) plus folic acid in each group are reported in Table 5:

TABLE 2a
Total Folates in Serum

| | Non- | Form | Control | FRAb + | |
|---|---|---|---|---|---|
| Serum | pregnant | Only | Ab + Form | Form | Baseline |
| PGA | 24.4 | 130.7 | 75.8 | 254.2 | |
| 5-methyl-(6S)- | 60.0 | 85.5 | 143.3 | 190.9 | |
| THF | | | | | |
| Folinic acid | 22.5 | 140.4 | 99.9 | 125.5 | |
| 5-formyl-(6S)- | 27.5 | 88.9 | 247.1 | 228.8 | |
| tetrahydrofolate | | | | | |
| Control | | | | | 153.0 |

### Observations:

1. 5-formyl-(6S)-tetrahydrofolate surprisingly produces superior concentrations of total folates especially in pregnant rats injected with the folate receptor antibody.

Additional analyses were performed in various tissues of the pregnant and non-pregnant rats. For tissues other than serum, each tissue was collected, weighed and stored at −20 C. For the folate assay each tissue was suspended in 3 volumes of phosphate buffer containing 2% ascorbic acid pH 7.4 and homogenized using a polytron probe. An aliquot of this homogenate was diluted 10 fold in PO4 buffer PH 5.5 containing 0.2% ascorbic acid, in a glass tube, sealed and placed in a boiling water bath for 10 min. The tube was cooled to room temperature, precipitate removed by centrifugation and the clear supernatant assayed for folate. Concentrations of total 5-MTHF (L- and D-) plus PGA in analyzed tissues are reported in Tables 6-10. Brain concentrations were derived from cortex and cerebellum.

TABLE 2b
Total 5-MTHF + PGA in Tissues of Non-Pregnant
Rats Following Administration of Four Folate
Forms (without antibody administration)

| | | 5-methyl- | | 5-formyl- |
|---|---|---|---|---|
| | PGA | (6S)-THF | Folinic acid | (6S)-THF |
| Liver | 56.0 | 75.4 | 104.2 | 93.0 |
| Kidney | 27.9 | 36.4 | 24.6 | 20.4 |
| Ovary | 11.3 | 7.2 | 6.8 | 9.8 |
| Brain | 3.1 | 1.9 | 3.9 | 4.8 |
| Cortex | 2.6 | 1.9 | 2.0 | 1.5 |
| Cerebellum | 3.5 | 1.9 | 5.9 | 8.2 |

TABLE 2c
Total 5-MTHF + PGA in Tissues of
Pregnant Rats Following PGA Administration

| | PGA | | |
|---|---|---|---|
| Tissue | PGA | PGA + | PGA + |
| (ng/mg) | (w/o IgG) | Control IgG | FRAb |
| Liver | 61.0 | 166.0 | 145.2 |
| Kidney | 25.5 | 81.6 | 29.6 |
| Ovary | 7.4 | 14.8 | 12.4 |
| Brain | 2.0 | 2.0 | 1.7 |
| Placenta | 5.7 | 2.9 | 2.4 |
| Embryo | 4.8 | 1.7 | 2.3 |

TABLE 2d
Total 5-MTHF + PGA in Tissues of Pregnant
Rats Following 5-methyl-(6S)-THF Administration

| | 5-methyl-(6S)-THF | | |
|---|---|---|---|
| | 5-methyl- | 5-methyl- | 5-methyl- |
| Tissue | (6S)-THF | (6S)-THF + | (6S)-THF + |
| (ng/mg) | (w/o IgG) | Control IgG | FRAb |
| Liver | 58.6 | 148.5 | 148.0 |
| Kidney | 25.5 | 20.3 | 64.9 |
| Ovary | 6.6 | 14.9 | 27.0 |
| Brain | 2.4 | 3.2 | 3.7 |
| Placenta | 6.4 | 2.3 | 2.1 |
| Embryo | 4.6 | 2.0 | 1.9 |

TABLE 2e
Total 5-MTHF + PGA in Tissues of Pregnant
Rats Following Folinic acid Administration

| | Folinic acid | | |
|---|---|---|---|
| Tissue | Folinic acid | Folinic acid + | Folinic acid + |
| (ng/mg) | (w/o IgG) | Control IgG | FRAb |
| Liver | 44.8 | 40.8 | 32.4 |
| Kidney | 20.2 | 10.8 | 16.7 |
| Ovary | 9.3 | 4.3 | 4.4 |
| Brain | 2.0 | 2.6 | 3.0 |
| Placenta | 7.6 | 5.1 | 3.5 |
| Embryo | 5.3 | 2.5 | 4.0 |

TABLE 2f
Total 5-MTHF + PGA in Tissues of Pregnant
Rats Following 5-formyl-(6S)-THF Administration

| | 5-formyl-(6S)-THF | | |
|---|---|---|---|
| | 5-formyl- | 5-formyl- | 5-formyl- |
| Tissue | (6S)-THF | (6S)-THF + | (6S)-THF + |
| (ng/mg) | (w/o IgG) | Control IgG | FRAb |
| Liver | 44.8 | 38.2 | 39.1 |
| Kidney | 20.2 | 19.8 | 14.3 |
| Ovary | 9.3 | 7.1 | 3.2 |
| Brain | 2.0 | 4.8 | 3.5 |
| Placenta | 9.0 | 3.3 | 3.0 |
| Embryo | 7.2 | 2.8 | 2.3 |

### Observations:

1. In non-pregnant rats without the folate receptor antibody, 5-formyl-6S-THF surprisingly results in much better concentrations of folates in ovary, brain and cerebellum than 5-methyl-6S-THF, emphasizing the utility of this treatment in pre-natal supplementation and prevention of neural tube defects and other fetal developed disorders.

2. In non-pregnant rats without the folate receptor antibody, tissue uptake of the unnatural D-isomer in folinic acid is significant in the ovary, brain, cerebellum and cortex, resulting in a potentially unhealthy interference with the natural L-isomer.

3. In pregnant rats without the folate receptor antibody, 5-formyl-6S-THF produces much better folate concentrations in the placenta and embryo than either 5-methyl-6S-THF or folinic acid, again suggesting the utility of this treatment as pre-natal supplementation for the prevention of neural tube defects and other developmental disorders which originate during fetal development.

### Example 3: Tissue Distribution of Folates 24 h Following Oral Administration of Various Folate Forms to Rat Pups

To determine tissue distribution of orally administered folate forms in young rat pups and study the effect of folate receptor antibodies on this distribution. PND 21 rat pups weighing about 50 g were administered folate forms orally at a dose of 4 mg/kg. Animals were euthanized after 24 h and tissues collected for analyzing total methylfolate in each tissue to provide a measure of normal distribution. Additional pups were administered FR Ab (100 ug) IP; 24 h prior to oral administration of folate forms to determine if FR Ab would affect tissue uptake of folates and to identify the form of folate most effective in restoring brain uptake in the presence of FR Ab.

Results are reported in Table 3a.

TABLE 3a

| | | Folate (ng/mg) (mean) | | | |
|---|---|---|---|---|---|
| | | | | Cere- | |
| Code | Condition | Liver | Kidney | bellum | Cerebrum |
| AA | control | 124.5 | 46.1 | 10.9 | 4.0 |
| AB | PGA | 149.5 | 73.5 | 38.4 | 6.6 |
| ABb | PGA + FRAb | 159.8 | 66.8 | 20.7 | 8.3 |
| AC | 5 methyl | 158.5 | 86.6 | 26.1 | 4.3 |
| ACb | 5 methyl + FRAb | 250.34 | 116.4 | 60.7 | 3.8 |
| AD | 5-formyl | 91.8 | 50.5 | 48.2 | 10.3 |
| ADb | 5-formyl + FRAb | 156.9 | 60.4 | 76.6 | 13.6 |
| AE | 5-formyl-6S | 80.0 | 52.1 | 93.3 | 15.3 |
| AEb | 5-formyl-6S + FRAb | 133.44 | 48.7 | 69.5 | 13.2 |

### Observations:

1. 5-formyl-6S-THF produces much higher folate concentrations in the cerebellum and cerebrum than 5-methyl-6S-THF in rat pups with and without the antibody, supporting the utility of the treatment when administered to children during neurodevelopment.

2. When folinic acid is administered to antibody-infected pups, an increase in folates in the brain is observed, corresponding to increased uptake of the unnatural D-isomer. The opposite is observed when 5-formyl-6S-THF is administered indicating that D-folates are preferentially taken up through the reduced folate carrier in response to the antibody, potentially interfering with the utilization of the natural L-isomer.

### Example 4: Exemplary Formulations

An exemplary liquid solution for administering in drop-wise fashion according to the present invention is described below in Table 4a:

TABLE 4a

| Ingredient | Concentration |
|---|---|
| 5-formyl-(6S)-THF, Ca²⁺ salt (pentahydrate) | 25 mg/ml* |
| Sodium gluconate USP | 40 mcg/ml |
| Water | q.s. |
| pH | 7.8 ± 1.0 |

*based on weight of anhydrous free acid

An exemplary film strip formulation is also provided. Using the components described in Table 4b laminate with a nominal size of 2,000 cm² can be obtained. From the dried laminate, films with a suitable size for delivering the correct dose can be punched.

TABLE 4b

| | | Other | |
|---|---|---|---|
| Active Ingredient | Polymers | components | Solvents** |
| 4.167 g | 7.333 g poly(vinyl | 0.200 g | 17.6 g |
| 5-formyl-6S- | alcohol) 4-88 | neohesperidin | ethanol 96% |
| THF Ca²⁺ * | | | (v/v) |
| | 2.000 g | 0.333 g | 2.3.3 g |
| | PEG 1000 | polysorbate 80 | purified water |
| | | 3.333 g | |
| | | rice starch | |

*pentahydrate; weight based on anhydrous free acid
**Removed during evaporation/curing

### Example 5. Pharmacokinetics of the 12.5 Mg Dose in Example 4

Based on pharmacokinetics reported for a 25 mg liquid elixir of d,l-leucovorin by McGuire 1988 (reproduced below in Table 5), and the pharmacokinetics of the liquid formulation of the present invention reported in Examples 1-3, the 12.5 mg dose of 5-formyl-6(s)-tetrahydrofolate in an alcohol-free aqueous vehicle=as described in Example 4 will produce an absolute bioavailability of reduced l-folates exceeding 94%, a cₘₐₓ of total reduced l-folates greater than 400 or 410 ng/ml, a cₘₐₓ of l,5-methyl-THF greater than 375 or 385 ng/ml, a cₘₐₓ of 5-Formyl-THF less than 40 or 30 ng/ml, and a ratio of 5-methyl-THF to 5-formyl-THF in excess of 10:1, using the methods of analysis and calculations reported by McGuire (1988) Table 1. In all of these measures, the formulations of the present invention will produce a statistically significant improvement in the pharmacokinetics reported of the formulation relative to the pharmacokinetics reported by McGuire 1988.

TABLE 5

| | Cₘₐₓ | tₘₐₓ | t½ | AUC_{0-24 h} | AUC_{0-inf} |
|---|---|---|---|---|---|
| | (ng/ml) | (min) | (hour) | (ng · h/ml) | (ng · h/ml) |
| Total folates^{a} | 393 ± 96 | 140 ± 32 | 6.0 ± 1.9 | 2063 ± 587 | 2149 ± 612 |
| 5-Formyl-THF^{b} | 51 ± 50 | 73 ± 35 | 9.5 ± 4.6 | 135 ± 80 | 140 ± 81 |
| 5-Methyl-THF^{c} | 367 ± 87 | 142 ± 29 | 6.0 ± 1.9 | 1929 ± 542 | 2012 ± 567 |

^{a}l-casei activity
^{b}Strep. faecalis activity
^{c}arithmetic difference between a and b, as described in McGuire (1988)

### Example 6: Clinical Study Evaluating Communication Improvements from 5-Formyl-(6S)-THF in Children with Communication Deficits

This study aims to examine the efficacy of a preservative-free liquid solution of 5-formyl-(6S)-THF administered from a preservative-free multi-dose eye dropper at a dose of 0.5 mg/kg/day or 1 mg/kg/day divided into two separate daily doses, with a maximum dose of 12.5 or 25 mg/day. The non-preserved aqueous formulation has a concentration of 25 mg/ml and will be dispensed in 40 mcl drops. Depending on the weight of the child, from 3 to 12 drops will be dispensed into a spoon, a small glass of water, juice or applesauce each administration period.

### Inclusion Criteria

Diagnosis of ASD based upon an assessment tool that utilizes the DSM-5 criteria (e.g., Autism Symptom Rating Scale (ASRS), Childhood Autism Rating Scale, Second Edition (CARS-2), or checklist, and confirmed with the ADOS-2
Between 2.5 and 5 years of age (inclusive)
Language impairment (Ages and Stage Questionnaire between −1 and −3 SD for Language or PLS-5 scaled score ≤85)
Moderate level of autism symptoms (Diagnosed on ADOS-2)

### Exclusion Criteria

Mineral or vitamin supplementation that exceeds the Tolerable Upper Daily Intake Levels set by the Institute of Medicine
Significant self-abusive or violent behavior or evidence of suicidal ideation, plan or behavior
Severely low functioning (i.e., having a MSEL cognitive index <55)
Severe prematurity (<34 weeks gestation) as determined by medical history

TABLE 6a
Schedule of Procedures

| Table 1. Schedule | | | | | | | | | | | |
|---|---|---|---|---|---|---|---|---|---|---|---|
| of Procedures | | Double blind Phase (weeks) | | | | | | | Open Label (wks) | | |
| Measure | Screen | 0 | *2 | *4 | 6 | *8 | *10 | 12 | *16 | *20 | 24 |
| Psychometrician | | | | | | | | | | | |
| ADOS-2 | X | | | | | | | | | | |
| BOSCC | | X | | | X | | | X | | | X |
| Expressive Language MSEL | X | X | | | X | | | X | | | |
| Receptive Language MSEL | X | X | | | X | | | X | | | |
| Cognition - MSEL | X | X | | | X | | | X | | | X |
| Clinician | | | | | | | | | | | |
| CGI-Language Ability | X | X | | | X | | | X | | | |
| CGI-LA | | | | | | | | | | | |
| CGI-Verbal Communication | X | X | | | X | | | X | | | |
| CGI-VC | | | | | | | | | | | |
| CGI-Improvement | | | | | X | | | X | | | X |
| OACIS | | X | | | X | | | X | | | X |
| Parent or Caregiver Questionnaire | | | | | | | | | | | |
| Ages and Stages Questionnaire | X | | | | | | | | | | |
| Vineland III Checklist Form | | X | | | | | | X | | | X |
| ABC | X | | | | X | | | X | | | X |
| CaGI-Language Ability | X | X | | | X | | | X | | | |
| CaGI-LA | | | | | | | | | | | |
| CaGI-Verbal Communication | X | X | | | X | | | X | | | |
| CaGI-VC | | | | | | | | | | | |
| CaGI-Improvement | | | | | X | | | X | | | |
| Parent Target Problems | X | | | | X | | | X | | | X |

### Efficacy Analysis

### Primary Objective:

To determine the efficacy and tolerability of L-leucovorin calcium treatment for expressive language in young children with ASD and language impairment (Age 2.5 to 5 years, inclusive)

### Secondary Objectives:

To measure proportion of patients achieving meaningful Improvement in Language Skills
To evaluate ASD phenotypes (based on age, anxiety, intellectual functioning and disease severity) most likely to benefit from treatment with levoleucovorin
To evaluate the effect of levoleucovorin compared to placebo on reducing repetitive and restrictive behaviors in ASD
To evaluate the effect of levoleucovorin compared to placebo on reducing aberrant behaviors in ASD, including hyperactivity, agitation, and irritability Results from the studies will be analyzed according to the following endpoint hierarchy:

### Primary Endpoint:

Change from baseline on the MSEL expressive language domain

### Secondary Endpoints:

Change from baseline on the MSEL receptive language domain
Proportion of participants achieving meaningful improvement as derived by the meaningful change threshold on the MSEL language domains

### Exploratory Endpoints:

Change from baseline on the ABC domains
Categorical change from baseline on the Clinician Global Impression of Verbal Communication (CGI-VC)
Categorical change from baseline on the Clinician Global Impression of Language Ability (CGI-LA)
Categorical change from baseline on the Caregiver Global Impression of Verbal Communication (CaGI-VC)
Categorical change from baseline on the Caregiver Global Impression of Language Ability (CaGI-LA)
Proportion of participants with at least “minimal improvement” in the CGI-C verbal communication domain
Proportion of participants with at least “minimal improvement” in the CGI-C language communication domain
Proportion of participants with at least “minimal improvement” in the CaGI-C verbal communication domain
Proportion of participants with at least “minimal improvement” in the CaGI-C language communication domain

### REFERENCES CITED

Spector R. Micronutrient Homeostasis in Mammalian Brain and Cerebrospinal Fluid. J. Neurochem., Vol. 53, No. 6, 1989:1667-74.
Ramaekers V T, Hausler M, Opladen T, et al. Psychomotor retardation, spastic paraplegia, cerebellar ataxia and dyskinesia associated with low 5-methyltetrahydrofolate in cerebrospinal fluid: a novel neurometabolic condition responding to folinic acid substitution. Neuropediatrics 2002; 33:301-8.
Stover P J, Durga J, Field M S. Folate nutrition and blood-brain barrier dysfunction. Current Opinion in Biotechnology 2017, 44:146-152.
Rezk B M, Haenena G R, van der Vijgh W J, Bast A. Tetrahydrofolate and 5-methyltetrahydrofolate are folates with high antioxidant activity. Identification of the antioxidant pharmacophore. FEBS Letters 555 (2003) 601-605.
United States Food and Drug Administration, FDA pharmacology review for Fusilev® (NDA 20-140)
Bertrand R, Jolivet J. Lack of interference by the unnatural isomer of 5-formyltetrahydrofolate with the effects of the natural isomer in leucovorin preparations. J Natl Cancer Inst 1988; 81: 1175-8.
Berrocal-Zaragoza M I, Fernandez-Ballart J D, Murphy M M, et al. Association between blocking folate receptor autoantibodies and subfertility. Fertil Steril 2009; 91(4 Suppl): 1518-1521.
Molloy A M, Quadros E V, Sequeira J M et al. Lack of Association between Folate-Receptor Autoantibodies and Neural-Tube Defects. N Engl J Med. 2009; 361:152-60.
Hyland, K., Surtees, R. Measurement of 5-methyltetrahydrofolate in cerebrospinal fluid using HPLC with coulometric electrochemical detection. Pteridines 1992, 3, 149-150.
McGuire B W, Sia L L, Leese P, Gutierrez M L, Stokstad E L R. Pharmacokinetics of leucovorin calcium after intravenous, intramuscular, and oral administration. Clin Pharmacol 1988; 7:52-58.
United States Food and Drug Administration, Population Pharmacokinetics Guidance for Industry, July 2019.

### Other Embodiments

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A method of making clinically meaningful improvements in MSEL expressive communication scores in a child less than 6 years of age, wherein the child is characterized by:
a) a moderate level of autism under ADOS-2, and
b) a normal CSF folate concentration of from 39.7 nmol/l to 174 nmol/l;
comprising orally administering to the child from 5 to 25 35-60 mcl drops per day of a pharmaceutical formulation comprising a preservative-free liquid solution of 5-formyl-6(s)-tetrahydrofolate or a pharmaceutically acceptable salt thereof, at a concentration of from 20 to 30 mcg/ml, that has an absolute bioavailability of 5-formyl-6(s)-tetrahydrofolate greater than or equal to 92%.

2) A method of making clinically meaningful improvements in MSEL expressive communication scores in a child less than 6 years of age having a developmental or chronic cerebral nervous system disorder comprising orally administering to the child a pharmaceutical formulation comprising a therapeutically effective amount of 5-formyl-6(s)-tetrahydrofolate or a pharmaceutically acceptable salt thereof.

3) (canceled)

4) (canceled)

5) (canceled)

6) The method of claim 1, wherein the child is less than sixty months of age.

7) The method of claim 1, wherein the child has a developmental or chronic cerebral nervous system disorder selected from:
a) autism based upon DSM-5 criteria, severity level 1, 2, or 3;
b) language impairment corresponding to from 1 to 3 standard deviations below the mean on ASQ-3 for communication;
c) language impairment corresponding to a scaled score ≤90 or 85 based on PLS-5 normative data reported in 1-month increments for children ages 2:6-2:11.
d) intellectual impairment corresponding to an composite standard score on the MSEL of ≤90 or 85;
e) language impairment corresponding to an expressive language standard score on the MSEL of ≤90 or 85;
f) epilepsy defined by abnormal EEG activity, anti-epileptic medication, or a history of seizures;
g) a moderate level of autism symptoms on ADOS-2; or
h) a combination thereof.

8) (canceled)

9) (canceled)

10) (canceled)

11) (canceled)

12) (canceled)

13) The method of claim 1, wherein the formulation when orally administered preferentially increases concentrations of tetrahydrofolate compared to a comparable formulation of 5-formyl-6(r,s)-tetrahydrofolate.

14) The method of claim 1, wherein the formulation comprising 12.5 mg of the 5-formyl-6(s)-tetrahydrofolate or a pharmaceutically acceptable salt thereof has been shown to produce a pharmacokinetic response selected from:
a) an absolute bioavailability of total reduced l-folates exceeding 92%, 93%, 94%, 95%, or 96%;
b) a cₘₐₓ of total reduced l-folates greater than 410 ng/ml, 420 ng/ml, 440 ng/ml, or 460 ng/ml;
c) a cₘₐₓ of 1,5-methyl-THF greater than 385 ng/ml, 400 ng/ml, 415 ng/ml, or 430 ng/ml;
d) a cₘₐₓ of 5-formyl-THF less than 40 ng/ml, 30 ng/ml, 20 ng/ml, or 10 ng/ml;
e) a ratio of 5-methyl-THF to 5-formyl-THF in excess of 10:1, 20:1, 50:1, 75:1, or 100:1; or
f) a combination thereof.

15) The method of claim 1, wherein the formulation is an alcohol-free aqueous solution at a pH of 7.8±1.0.

16) (canceled)

17) (canceled)

18) (canceled)

19) (canceled)

20) (canceled)

21) (canceled)

22) The method of claim 1, wherein the child is suffering from imbalanced, perturbed, or impaired neurogenesis or tetrahydrofolate utilization.

23) The method of claim 1, wherein the child is suffering from imbalanced, perturbed, or impaired tetrahydrofolate utilization in a tissue selected from the cerebellum, cortex, placenta, embryo, or ovary, and the method preferentially increases concentrations of tetrahydrofolate in one or more of the tissues.

24) The method of claim 1, wherein the child is suffering from impaired neurogenesis selected from neuronal synthesis, neuronal integration, neuronal differentiation, abnormal axonal branching and impaired myelin synthesis, and the method improves the neurogenesis.

25) (canceled)

26) The method of claim 1, wherein the therapeutically effective amount comprises from about 0.1 to about 1.0 mg/kg/day, up to a maximum of 25 mg/day, based on the weight of the anhydrous free acid of the 5-formyl-(6S)-tetrahydrofolate.

27) (canceled)

28) The method of claim 1, wherein the therapeutically effective amount comprises from about 5 to about 25 mg/day, based on the weight of the anhydrous free acid of the 5-formyl-(6S)-tetrahydrofolate.

29) (canceled)

30) (canceled)

31) The method of claim 1, wherein the child is on aripiprazole or risperidone or a pharmaceutically acceptable salt thereof.

32) The method of claim 1, wherein the formulation is preservative free and is administered from a preservative free dropper restricts the ingress of contaminants from the surrounding air to the solution during use and the regress of the liquid solution that has been dispensed from the dropper.

33) A drug product comprising a multi-dose dropper comprising from 5 to 50 ml of an aqueous oral solution of 5-formyl-(6S)-tetrahydrofolate or a pharmaceutically acceptable salt thereof, wherein the concentration of 5-formyl-(6S)-tetrahydrofolate in the solution is from 20 to 30 mg/ml based on the weight of the anhydrous free acid, and each drop dispensed from the dropper comprises from 35 to 150 microliters of solution.

34) The drug product of claim 33, wherein the oral solution is sterile and preservative free.

35) The drug product of claim 33, wherein the dropper restricts the ingress of contaminants from the surrounding air to the solution during use and the regress of the liquid solution that has been dispensed from the dropper.

36) The drug product of claim 33, wherein the solution comprises from 20 to 30 mg/ml of 5-formyl-(6S)-tetrahydrofolate based on the weight of the anhydrous free acid, and the dropper dispenses from 35 to 60 microliters per drop.

37) (canceled)

38) The drug product of claim 33, wherein the solution consists essentially of water, 5-formyl-(6S)-tetrahydrofolate calcium, sodium gluconate, and optionally sodium hydroxide or hydrochloric acid, at a pH of from 6.8 to 8.8.

39) (canceled)
